(19) United States
(12) Patent Application Publication
(10) Pub. No.: US 2016/0287822 A1
(43) Pub. Date: Oct. 6, 2016

(54) Systems and Methods for Detection of Respiratory Events Using Pattern Recognition

(71) Applicant: Milenko Masic, Carlsbad, CA (US)

(72) Inventor: Milenko Masic, Carlsbad, CA (US)

(21) Appl. No.: 15/086,621

(22) Filed: Mar. 31, 2016

Related U.S. Application Data
(60) Provisional application No. 62/143,098, filed on Apr. 4, 2015.

(51) Int. Cl. A61M 16/00

(52) U.S. Cl. CPC A61M 16/0003; A61M 2016/003; A61M 2230/005; A61M 2230/40

(57) ABSTRACT

Various embodiments of the present invention provide systems, methods and devices for respiratory support. As one example, a method is described that includes providing a measured signal such as gas pressure or flow or any respiratory signal derived or obtained by combining pressure or flow signals related to a patient's breathing. Breathing events of interest are detected by establishing similarity of at least a portion of the measured signal with a reference signal or a collection of reference signals using a similarity criterion. A ventilation cycle is triggered if patients breath phase transition event is detected from the measured signal using the described method.

### CROSS REFERENCE TO RELATED APPLICATIONS

This application claims the benefits of U.S. Provisional Application No. 62143098 filed on Apr. 4, 2015, titled “Systems and Methods for Detection of Respiratory Events Using Pattern Recognition” and whereby the complete disclosure of such application is hereby incorporated by reference.

### BACKGROUND

The present invention is related to respiratory devices, such as but not limited to mechanical ventilators, patient monitoring devices, and diagnostics devices that detect events in a patient's breathing.

Mechanical ventilators are used to ventilate lungs of those patients whose ability to breathe normally is impaired. Respiratory support by mechanical ventilators is achieved by providing different breathing gas mixtures to the patient using various ventilation modes typically adjusted to the patient's individual treatment needs. Successful ventilation is often dependent and related to the ability of the ventilator to detect different events in the patient's breathing, and to deliver breath accordingly. This includes the ability of the ventilator to synchronize the breath delivery with the beginning and end of the patient's breath.

Currently there exist methods for detection of breathing events most of which employ techniques to directly compare the values of the used signals to certain thresholds that indicate the presence of the events of interest. These methods typically use only the absolute value of the measured signal, and are incapable of detecting characteristic changes of the signal associated with breathing events when the signal is away from the threshold.

For example, certain methods use the pressure signal measured in the patient ventilator interface for the detection of the onset of the patient's inspiration. These methods detect when the pressure signal drops below the pressure baseline maintained by the mechanical ventilator during the exhalation phase of a breath. However, the patient may start a new inhalation before the current exhalation has completed and the breathing gas inhaled during the previous inhalation has been fully exhaled and the pressure in the interface has decayed to the baseline value. In this case, the delay between the actual onset of the patient's inhalation and the moment when the algorithm detects it is caused by the additional time needed for the pressure in the patient-ventilator interface to first drop to the baseline and then below the detection threshold. Depending on the strength of the patient effort, this delay can be substantial and represent a significant shortcoming. Moreover, the pressure in the interface may completely fail to reach the baseline and the detection threshold in the case of weak breathing efforts causing that the actual breath remains completely undetected.

The occurrence of these wasted efforts or their significantly delayed detections are very unpleasant for ventilated patients. They affect their comfort, increase the work of breathing, may tire the patient and result in prolonged treatments and often a need for sedation.

Patient's inspiratory efforts, regardless of how big or small they are, cause deviations in the pressure signal before it reaches its exhalation baseline, and they can be detected if suitable methods are used. These methods, however, do not rely only on the absolute value of the measured signal to detect events of interest, but they are designed to detect characteristic changes in the profile of the signal used for detection regardless of its actual range. The invention described here enables detection of breathing events using methods to detect presence of predefined profiles in samples of the measured respiratory signals.

Hence, there exists a need in the art for respiratory event detection methods based on pattern recognition for ventilation systems and respiratory devices, and methods for using such.

### BRIEF SUMMARY

The present invention is related to respiratory devices, such as but not limited to mechanical ventilators, patient monitoring devices, and diagnostics devices that detect events in a patient's breathing.

Some embodiments of the present invention provide methods for respiratory support. Such methods include providing a measured signal such as gas pressure or flow or any respiratory signal derived or obtained by combining pressure or flow signals related to patients breathing. The measured respiratory signal is compared to a provided reference signal and a degree of similarity between the measured respiratory signal and the reference signal is determined using an adopted similarity criterion. The determined degree of similarity between the measured respiratory signal and the reference signal is utilized for respiratory support.

In one or more instances of the aforementioned embodiments, utilizing the determined degree of similarity between the measured respiratory signal and the reference signal for respiratory support includes detection of breathing events such as onsets of patients inspiration, and initiation of the ventilation cycle based at least in part on the detected event.

In other instances of the aforementioned embodiments, utilizing the determined degree of similarity between the measured respiratory signal and the reference signal for respiratory support includes detection of an onset of a patient's exhalations, and termination of the ventilation cycle based at least in part on the detected event.

Other embodiments of the present invention provide respiratory support systems that include a processor communicably coupled to a computer readable medium. The computer readable medium includes software and/or firmware that is executable by the processor to: receive a measured respiratory signal, receive a reference signal. The software and/or firmware is further executable to determine a degree of similarity between the measured respiratory signal and the reference signal using an adopted similarity criterion. The software and/or firmware is further executable to use the determined degree of similarity between the measured respiratory signal and the reference signal. In one particular embodiments of the present invention the software and/or firmware may use the determined degree of similarity between the measured respiratory signal and the reference signal to affect the course of the treatment of the subject that uses the said respiratory support system. In another embodiment of the present invention the software and/or firmware may use the determined degree of similarity between the measured respiratory signal and the reference signal by making this information available to the user via a display.

Yet other embodiments of the present invention provide patient ventilators that include a gas inlet, a gas outlet, a tube coupling the gas inlet and the gas outlet, a sensor operable to provide a measured respiratory signal. In addition, the ventilators include a processor communicably coupled to a computer readable medium with instructions executable by the processor to: receive a measured respiratory signal, receive a reference signal. The software and/or firmware is further executable to determine a degree of similarity between the measured respiratory signal and the reference signal using an adopted similarity criterion. The software and/or firmware is further executable to use the determined degree of similarity between the measured respiratory signal and the reference signal.

This summary provides only a general outline of some embodiments of the invention. Many other objects, features, advantages and other embodiments of the invention will become more fully apparent from the following detailed description, the appended claims and the accompanying drawings.

### DESCRIPTION OF THE DRAWINGS

A further understanding of the various embodiments of the present invention may be realized by reference to the figures which are described in the remaining portions of the specification.

FIG. 1 depicts one possible shape of the reference signal whose presence in a segment of a respiratory signal may be monitored to detect related respiratory events in accordance with various embodiments of the present invention;

FIG. 2 depicts how moving windows, occupying positions w₁, w₂, and w₃ as time, t, elapses, may be used to extract data from the respiratory signal to analyze them for presence of patterns of interest in accordance with some embodiments of the present invention;

FIG. 3 depicts a segment of a respiratory signal, s(t), captured during time window, that exhibits a low degree of similarity with the shown reference signal, r(t), according to certain embodiments of the present invention;

FIG. 4 depicts a segment of a respiratory signal, s(t), captured during time window, w₃, that exhibits a high degree of similarity with the shown reference signal, r(t), according to certain embodiments of the present invention;

FIG. 5 depicts how a segment of a respiratory signal, s(t), is modified to create modified respiratory signal, s^{M}(t), so that the first part of the modified respiratory signal, s^{M}(t), is aligned with the first part of the reference signal, r(t), using an alignment method and thereby prepared for comparison with the shown reference signal, r(t), in accordance with some embodiments of the present invention;

FIG. 6 provides graphical results of a numerical simulation of certain embodiments of the present invention illustrating how the present invention can be used to detect onsets of inspirations of a ventilated patients by detecting a presence of related patterns in the flow signal, and how it can be used to pace the ventilator's breath delivery through implementation of particular embodiments of the present invention;

### DETAILED DESCRIPTION

Various embodiments of the present invention provide systems and methods for the detection of respiratory events with a reasonable accuracy that are based on the detection of characteristic patterns in a monitored signal affected by breathing.

In some embodiments of the present invention the fact that a respiratory event has been detected is used by the respiratory support system to affect the respiratory support system's behavior which includes but is not limited to initiation of breath phase transitions.

The detection of respiratory events in accordance with the present invention is performed by providing a measured respiratory signal, a reference signal that is intended to describe the expected patterns in the measured respiratory signal indicative of the presence of the respiratory events of interest, and by determining a degree of similarity between the measured respiratory signal, and the adopted reference signal using an adopted similarity criterion. The information obtained from this process is used for respiratory support of a patient in various ways including delivery of a breath by a mechanical ventilator or other respiratory devices once an inspiration is detected, or to terminate gas delivery to the patient once the beginning of exhalation is detected.

Various embodiments of the present invention are possible. The presentation in the sequel outlines how the present invention may be realized in certain particular embodiments.

The shape of a reference signal is adopted to reflect the patterns in the processed respiratory signal that are expected to occur when related respiratory events occur.

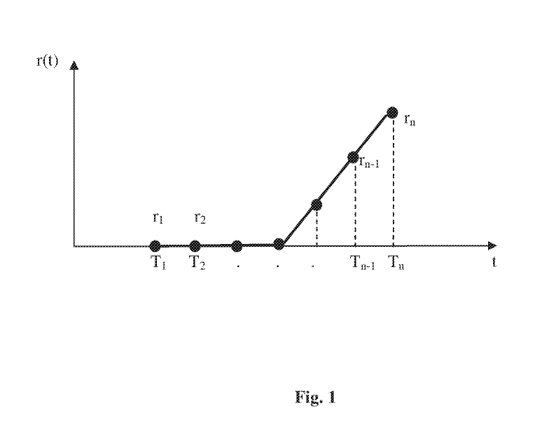

For example, FIG. 1 depicts a reference signal that with some embodiments of the present invention may be used to detect a sudden changes of the trend in the flow signal that is typically associated with the onset of the patient's inspiration. In other embodiments of the present invention different reference signals can be used for detection of other patterns in the observed signal if it is believed that their shape has a relationship to the respiratory events of interest.

Because the observed signal is compared with the reference in order to detect characteristic patterns, the shape of the reference signal is typically adopted such that it reflects the expected pattern that is to be detected.

In one embodiment of the invention the reference signal is defined and represented by n samples, collected at n points in time, T₁, T₂, . . . , Tₙ₋₁, as shown in FIG. 1, that are arranged in the vector r consisting of n components as follows:

$$r = [ \begin{matrix}{r}_{1} \\ … \\ {r}_{n}\end{matrix} ] .$$

The measured respiratory signal is monitored for the presence of patterns of interest using various methods, some of which are described in more detail in the sequel. A detection of a respiratory event indicates that the related respiratory event has likely occurred.

The measured respiratory signal in which the presence of the pattern defined by the reference signal is monitored is sampled for a comparison with the reference signal.

Different embodiments of be present invention use different respiratory signals to detect respiratory events. Some of these respiratory signals may be measured directly, and some may be derived from the respiratory signals available for measurements. Flows and pressures of the breathing gas are some of the examples of the signals that can be used in certain embodiments of the present invention.

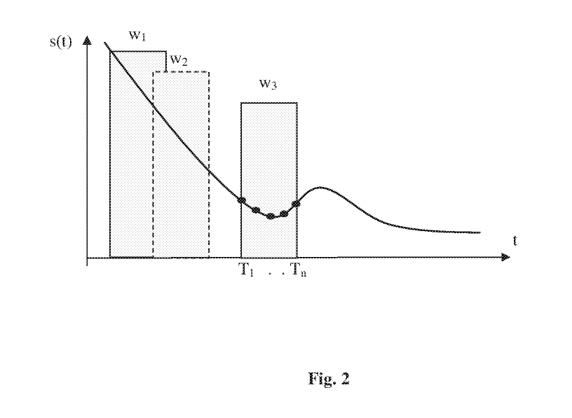

FIG. 2 depicts how a respiratory signal can be divided into separate windows for analysis and detection of the presence of respiratory events.

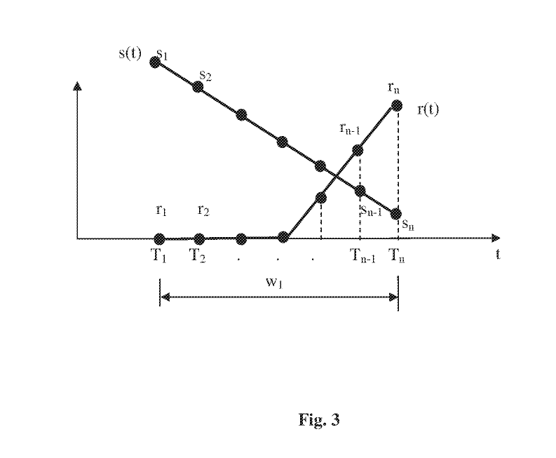

In one embodiment of the invention, a time window of the respiratory signal is selected, from which n samples are collected at n points in time, T₁, T₂, . . . , Tₙ₋₁, Tₙ, as shown in FIG. 3, and arranged in the vector s consisting of n components as follows:

$$s = [ \begin{matrix}{s}_{1} \\ … \\ {s}_{n}\end{matrix} ]$$

In one embodiment of the invention, a real-time detection of the presence of the event in the signal can be accomplished by moving the sampled window in time, and performing the event detection on the current time window that spans from some point in the past to the present time.

In one embodiment of the invention the time windows may overlap, while in another embodiment they may be disjoint.

Based on the sampled data from the time window, the respiratory signal is analyzed for the presence of the patterns of interest.

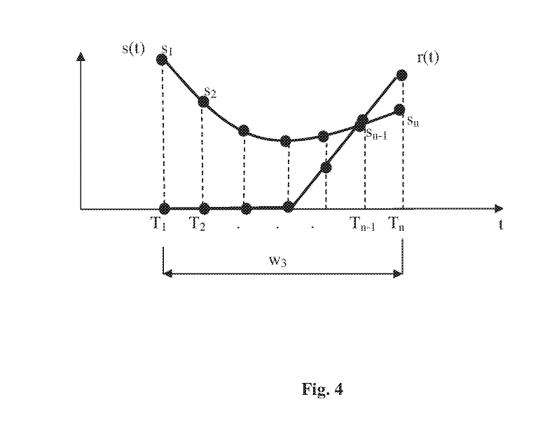

For example, if the respiratory signal s(t), within the analyzed time window contains that date similar to those depicted in FIG. 3 it is intuitively obvious that the respiratory signal within this window has very little similarity with the adopted reference signal, r(t), in FIG. 1 in which a change of the trend is evident, while in the respiratory signal it is absent. In contrast, FIG. 4 depicts a respiratory signal, s(t), that exhibits a change of the trend, and that is in that regard more similar to the reference signal r(t), in FIG. 1. In the sequel, a detailed and more specific description of some of the methods to measure the degree of similarity between the reference signal and the respiratory signal within the window are described.

In one embodiment, of the invention, the processed respiratory signal is first modified, before the degree of similarity with the reference signal is determined. As a result of such modification the original vector s is transformed into the vector that is denoted s^{M}.

$${s}^{M} = [ \begin{matrix}{s}_{1}^{M} \\ … \\ {s}_{n}^{M}\end{matrix} ]$$

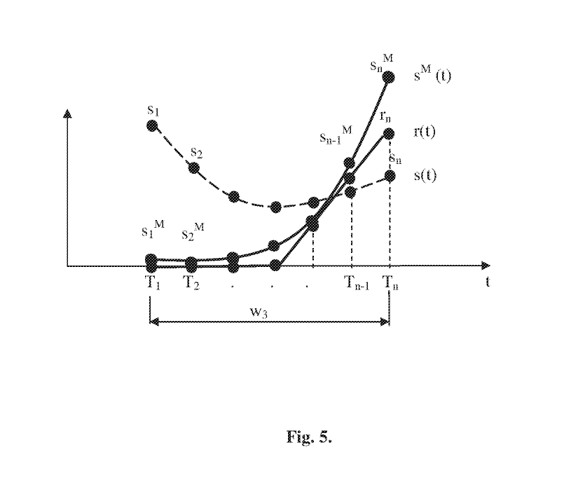

In one embodiment of the invention this modification may be used for example to achieve the alignment of the first portion of the processed signal with the first portion of the reference signal according to a certain alignment criteria FIG. 5 illustrates the main idea of this step, and how it results in two signals, r and s^{M} whose relationship is reasonably well captured by a linear regression. Note that if the signal s^{M} consisted of two straight line segments like the signal r shown does, linear regression would perfectly capture the relationship between them.

To present the main underlying idea of this invention it will be assumed, without a loss of generality, that the first portion of the adopted reference signal corresponds to a segment of a straight line, and that this first segment is aligned with the time axis as shown in FIG. 1 This assumption shall not limit the scope of this invention to reference signals with straight segments, but it is used only to facilitate the clarity of the presentation of the invention. It is well known to those skilled in the art that achieving similar equivalent objectives is possible when the first segment of the reference signal is not a segment of a straight line.

In one embodiment of this invention the alignment of the signals is achieved by fitting a straight line through the first part of the processed signal vector, and using this fitted line for detrending the entire vector of the processed respiratory signal. By doing so the first portion of the processed respiratory signal will be aligned with the time axis as shown in FIG. 5 according to certain alignment criteria.

In another embodiment of this invention the entire processed respiratory signal is moved as a rigid body to achieve the alignment of the first part of the signals. This movement for example can be achieved by finding and applying rotational and translational transformations of the signal yielding the desired alignment.

The degree of similarity between the processed respiratory signal and reference signal is determined after modifications to the processed respiratory signal are completed by computing a metric of the degree of their similarity.

Different methods to determine the degree of similarity between the reference signal and the modified respiratory signal are possible.

In one embodiment of this invention, the degree of similarity, between the respiratory signal, s^{M}, and reference signal, r, is determined by computing the magnitude of the projection of the normalized vector of the modified processed signal onto the normalized vector of the reference signal. The magnitude of this projection can for example be determined using to the following formula,

$$ρ = \frac{{\sum }_{i = 1}^{n} ( {r}_{i} - \overline{r} ) ( {s}_{i}^{M} - {\overline{s}}^{M} )}{\sqrt{{\sum }_{i = 1}^{n} {( {r}_{i} - \overline{r} )}^{2}} \sqrt{{\sum }_{i = 1}^{n} {( {s}_{i}^{M} - {\overline{s}}^{M} )}^{2 }}}$$

where ρ denotes the computed degree of similarity metric, and the mean values of the reference signal and modified processed signal are computed as:

$$\overline{r} = \frac{1}{n} {\sum }_{j = 1}^{n} {r}_{i} , \\ {\overline{s}}^{M} = \frac{1}{n} {\sum }_{j = 1}^{n} {s}_{j}^{M}$$

The computed degree of similarity p can be interpreted as the regression coefficient, or correlation coefficient between the vectors r and s^{M}.

In other embodiments of this invention, known statistical parameters indicating the goodness of the regression fit between the reference signal and the modified processed signal can be used as the similarity metric criteria. For example, F-value of the chi-square statistical distribution can be computed from the vectors r and s^{M} using the following formula:

$$F = \frac{\frac{{\sum }_{i = 1}^{n} ( ρ {r}_{i} - {\overline{s}}^{M} )}{{dof}_{regression}}}{\frac{{\sum }_{i = 1}^{n} ( {s}_{i}^{M} - ρ {r}_{i} )}{{dof}_{error}}}$$

where dof_{regression} and dofₑᵣᵣₒᵣ denote the degree of freedom parameters defined according to the standard statistical terminology and that are related to the number of samples n in the vectors r and s^{M}, and the number of coefficients of the linear regression.

It is not the intent of this document to present all possible ways that the degree of similarity between a reference signal and a processed signal can be measured. Those skilled in the art shall be able to define similar similarity metrics all of which shall be considered covered by this invention.

Once the degree of similarity between the adopted reference signal and the respiratory signal is determined this information can be used to judge the presence of the characteristic patterns defined by the adopted reference signal.

In one embodiment of the current invention a threshold value for the computed degree of similarity can be adopted so that when the computed degree of similarity exceeds the threshold the respiratory event is considered detected.

Upon a detection of a respiratory event, the respiratory system supporting the event detection algorithm responds according to a predefined policy. In one embodiment of the present invention, if onset of a patient inspiration is detected, a ventilator may start delivery of a breath to the patient. Similarly, if the end of an inspiration is detected, the ventilator may initiate its own exhalation routine. Detections of other respiratory events such as coughs are also possible using this invention.

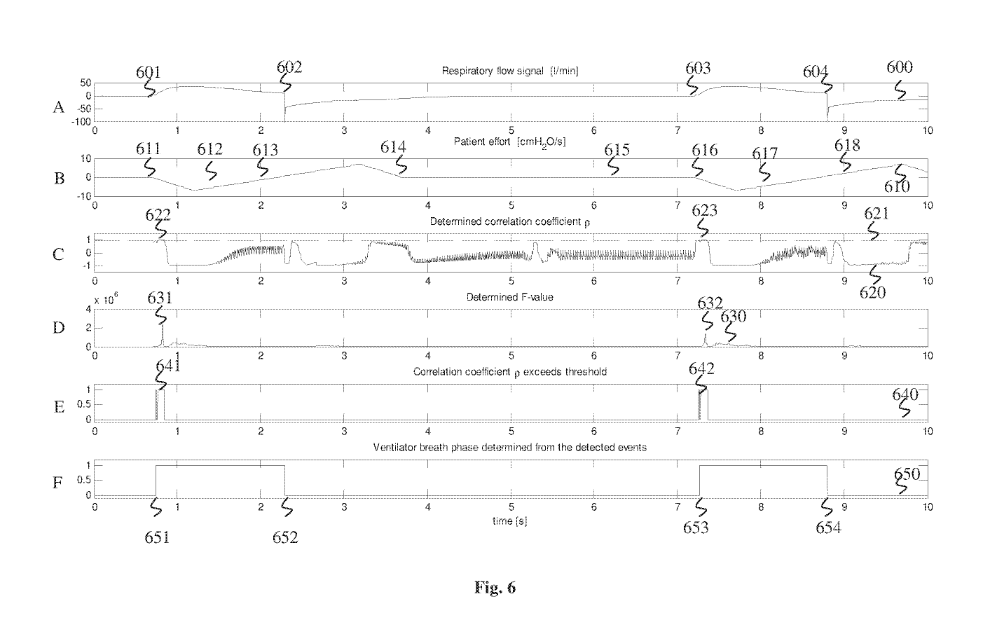

FIG. 6 depicts a simulated application of the invented method in a respiratory support system consisting of a mechanical ventilator to which an actively breathing patient is connected. The first trace from the top, FIG. 6A, shows the ventilator flow signal 600 in which the presence of the pattern shown in FIG. 1 is actively monitored in order to detect onsets of inspirations. The second trace from the top, FIG. 6B, depicts the periodic patients diaphragmatic effort 610 causing movements of air in the mechanical ventilator system typically characterized by the presence of the patterns 601 and 603 in the monitored flow signal 600 similar to the one shown in FIG. 1. The respiratory effort signal shown in the trace FIG. 6B has negative values 612 during inspirations, and becomes positive when the inspiration ends 613. The respiratory effort signal is equal to zero 615 between a completed exhalation 614 of one breath cycle and beginning of inspiration 616 of the subsequent breath cycle. The third trace, FIG. 6C, depicts the computed value of the correlation coefficient, ρ, 620 whose value exceeding the shown threshold 621 indicates that the pattern is detected in the flow signal. The trace FIG. 6D shows how the computed F-value 630 exhibits sudden increases 631 and 632 when the monitored pattern is present in the respiratory signal indicating how the F-value metric described in the invention can also be used to detect respiratory events as described in this invention. The high value of the signal 640 shown in the trace FIG. 6E corresponds to the intervals during which exists a significant similarity between the flow signal and the adopted reference signal. The rising edges 641 and 642 of this signal 640 is used to indicate onsets of detected inspirations that are in turn used to drive breath phase transitions 651 and 653 shown in the trace FIG. 6F. High values of the signal 650 in the bottom trace correspond to the inspiratory phase of the ventilator cycles while low values indicate exhalations. Observe how the high values 641 and 642 of the correlation coefficient 640 that exceed the set threshold coincide with the onsets 611 and 616 of inspirations that hence may be used as a method for detection of inspirations according to certain embodiments of the present invention.

This numerical simulation illustrates that the present invention may be used in respiratory support systems such as mechanical ventilators to detect respiratory events and affect breath phase transitions.

The present invention provides novel systems, methods, and devices for detection of respiratory events. While detailed descriptions of one or more embodiments of the invention have been given above, various alternatives, modifications, and equivalents will be apparent to those skilled in the art without varying from the spirit of the invention. Therefore, the above description should not be taken as limiting the scope of the invention, which is defined by the appended claims.

What is claimed is:

1. A method for respiratory support, the method comprising at least of:
providing a measured respiratory signal;
providing a reference signal;
determining a degree of similarity between the measured respiratory signal and the reference signal using an adopted similarity criterion;
utilizing the determined degree of similarity between the measured respiratory signal and the reference signal for respiratory support

2. The method of claim 1, wherein the measured respiratory signal is a measured signal that is sensitive to respiration.

3. The method of claim 1, wherein the measured respiratory signal is derived in part from some combination of at least one signal from a group consisting of: a measured pressure of the respired substance, a signal derived from or sensitive to the measured pressure, a measured flow of the respired substance, a signal derived from or sensitive to the measured flow, a measured temperature of the respired substance, a signal derived from or sensitive to the measured temperature, a measured concentration of at least one constituent of the respired substance, a signal derived from or sensitive to the measured concentration, and a measured signal that is sensitive to respiration.

4. The method of claim 1, wherein determining a degree of similarity between the measured respiratory signal and the reference signal using an adopted similarity criterion includes computing the correlation between the sample of the values obtained from the measured respiratory signal and the sample of values obtained from the reference signal.

5. The method of claim 1, wherein determining a degree of similarity between the measured respiratory signal and the reference signal using an adopted similarity criterion includes processing the measured respiratory signal through a filter matched to the adopted reference signal.

6. The method of claim 1, wherein determining a degree of similarity between the measured respiratory signal and the reference signal using an adopted similarity criterion includes statistical hypothesis testing if the data originate from the same distributions.

7. The method of claim 1, wherein utilizing the determined degree of similarity between the measured respiratory signal and the reference signal for respiratory support includes declaring an event detected if the degree of similarity between the measured respiratory signal and the reference signal satisfies a predefined criterion.

8. The method of claim 1, wherein utilizing the determined degree of similarity between the measured respiratory signal and the reference signal for respiratory support includes utilizing at least one of previously determined degrees of similarity between the measured respiratory signal and the reference signal.

9. The method of claim 1, wherein utilizing the determined degree of similarity between the measured respiratory signal and the reference signal for respiratory support includes detection of a respiratory event and initiating an action based at least in part on the fact that a respiratory event has been detected.

10. The method of claim 1, wherein utilizing the determined degree of similarity between the measured respiratory signal and the reference signal for respiratory support includes computing or using statistical parameters related to the probability or confidence of the determined degree of similarity.

11. The method of claim 9, wherein the detection of respiratory events includes but is not limited to the detection of the beginning and end of a breath, sigh, cough, or hick up.

12. The method of claim 9, wherein initiating an action based at least in part on the fact that a respiratory event has been detected includes a breath cycle transition of a respiratory support device such as but not limited to a mechanical ventilator.

13. A respiratory support system, the respiratory support system comprising:
a processor communicably coupled to a computer readable medium, wherein the computer readable medium includes instructions executable by the processor to:
receive a measured respiratory signal;
receive a reference signal;
determine a degree of similarity between the measured respiratory signal and the reference signal using an adopted similarity criterion;
use the determined degree of similarity between the measured respiratory signal and the reference signal

14. The system of claim 13, wherein to use of the determined degree of similarity between the measured respiratory signal and the reference signal includes affecting the course of the treatment of the subject that uses the said respiratory support system.

15. The system of claim 13, wherein to use of the determined degree of similarity between the measured respiratory signal and the reference signal includes making the determined degree of similarity between the measured respiratory signal and the reference signal known to a user of the respiratory support system.

16. The system of claim 15, wherein making the determined degree of similarity between the measured respiratory signal and the reference signal known to a user of the respiratory support system includes displaying it in a visual form.

17. The system of claim 15, wherein making the determined degree of similarity between the measured respiratory signal and the reference signal known to a user of the respiratory support system includes transferring the information to the user.

18. The system of claim 15, wherein the user of the respiratory support system is an operator of the respiratory support system.

19. The system of claim 15, wherein the user of the respiratory support system is an external device connected to the respiratory support system.

20. The system of claim 14, wherein affecting the course of a treatment of a subject that uses the said respiratory support system includes a change of an operational parameter of the respiratory support system.

21. The system of claim 14, affecting the course of a treatment of a subject that uses the said respiratory support system includes a decision to transition between breath cycles of the respiratory support system.

22. A patient ventilator, the ventilator comprising at least of:
a gas inlet;
a gas outlet;
a tube coupling the gas inlet and the gas outlet;
a sensor, wherein the sensor is operable to provide a measured respiratory signal;
a processor communicably coupled to a computer readable medium, wherein the computer readable medium includes instructions executable by the processor to:
receive a measured respiratory signal;
receive a reference signal;
determine a degree of similarity between the measured respiratory signal and the reference signal using an adopted similarity criterion;
use the determined degree of similarity between the measured respiratory signal and the reference signal to affect the course of the patients treatment during the respiratory support by the said ventilator system.
